(19) United States
(12) Patent Application Publication
(10) Pub. No.: US 2022/0079164 A1
(43) Pub. Date: Mar. 17, 2022

(54) DRY NONWOVEN ANTIBACTERIAL ARTICLE

(71) Applicants: TWE MEULEBEKE, Meulebeke (BE); HYGINN BVBA, Antwerp (BE)

(72) Inventors: Vincent DE SMEDT, Antwerp (BE); Dany MICHIELS, Haaltert (BE); Véronique DECAMBRAY, Waregem (BE); Sarah LEBEER, Mortsel (BE); Dieter VANDENHEUVEL, Leuven (BE)

(73) Assignees: TWE MEULEBEKE, Meulebeke (BE); HYGINN BVBA, Antwerp (BE)

(21) Appl. No.: 17/422,247

(22) PCT Filed: Jan. 24, 2019
(86) PCT No.: PCT/EP2019/051721

§ 371 Date: Jul. 12, 2021

(51) Int. Cl. A01N 63/22; A01N 25/34; A01N 63/20; A01P 1/00; B65D 83/08; A47L 13/17; A47L 13/51; B08B 1/00

(52) U.S. Cl. CPC A01N 63/22; A01N 25/34; A01N 63/20; B08B 1/006; B65D 83/0805; A47L 13/17; A47L 13/51; A01P 1/00

(57) ABSTRACT

The invention relates to the field of individual hygiene and in particular disinfecting nonwoven articles. A dry antibacterial article comprising at least a dispersible nonwoven layer is characterized in that unprotected and dry bacterial spores are spread within the nonwoven layer, said spores being selected to specifically inhibit the growth of pathogenic bacteria upon reactivation. The invention further relates to the use of the antibacterial article, a dispenser for wipes made from the article as well as manufacturing processes.

The invention relates to the field of individual hygiene and in particular disinfecting nonwoven articles.

There is a growing need for solutions to control hygiene in public places, in particular in public restrooms. Individuals want to make sure by themselves that their hygiene standards are met.

Some restrooms offer an access to disinfecting sprays or gels of chemical agents that can be applied to a wipe or to toilet paper and spread over the toilet seat to sterilize it. These solutions have however several drawbacks. Firstly, the disinfecting liquid spray or gel can leek from the wipe to the fingers of the user, which can be irritating to the skin. Secondly, the maintenance of restrooms being done at more or less long intervals can lead to a shortage of wipes and/or disinfecting spray or gel within the restroom. Finally, such products do not match the growing expectations of sustainability and environmental friendliness.

Disinfecting wet wipes, sold in sealed packages, which can easily be transported by individuals, are also widely spread on the market. However, these are not readily biodegradable once flushed. Indeed, as these are conditioned wet, they should not degrade in presence of humidity. Moreover, if the packet is not properly resealed after a wipe has been taken out, the rest of the wipes tend to dry and are therefore not usable anymore. The shelf life of these types of wipes is therefore limited.

Dry disinfecting wipes, disposable, biodegradable upon flushing are also described in WO2013171343. These wipes comprise two layers, a first layer of biodegradable paper, attached to a second layer of nonwoven material in which are dispersed microcapsules containing a cleansing agent. The paper layer is the handling side for the user. The nonwoven layer is the cleansing side. The microcapsules are expected to burst upon friction to release the cleansing agent in a liquid form. However, the encapsulation of a cleansing agent, be it a chemical agent or a probiotic agent, is technically challenging and the cost associated to the production of microcapsules is rather high. Moreover, the dispersion of such microcapsules in the nonwoven layer is also technically challenging, as a sufficient adherence of the microcapsules to the nonwoven material during transport is not easily achieved. These microcapsules are furthermore fragile and can be degraded during the dispersion step or during packaging and storing of the product.

There is therefore a real need to offer on the market a solution to the above mentioned drawbacks of the existing hygiene products.

It is the object of the present invention to propose a dry disinfecting wipe, which is dispersible in water, easily transportable with a long shelf life, producible at an attractive cost and offering simplicity of use to individuals wishing to control the hygiene conditions in public places like public restrooms.

### SOLUTION OF THE INVENTION

The present invention concerns a dry antibacterial article comprising at least a dispersible nonwoven layer characterized in. that unprotected and dry bacterial spores are spread in the nonwoven layer, said spores being selected to specifically inhibit the growth. of pathogenic bacteria upon reactivation.

The article of the invention is preferably flushable.

Advantageously, the dry antibacterial article also comprises a dispersible handling layer.

The invention also relates to the use, by a user, of the dry antibacterial article of the invention to clean a hard surface comprising the following steps of:
the user takes the dry antibacterial article in a hand,
the dry antibacterial article is humidified to initiate the reactivation of the spores,
the user wipes the hard surface with the article causing the release of spores onto the hard surface, and
the user throws the article away.

The invention further relates to a dispenser of wipes made with the dry article of the invention, said dispenser comprising:
a dry compartment comprising at least one wipe,
a wet compartment, comprising an. aqueous liquid,
means arranged to release an. aqueous liquid onto the wipe, and
a dispensing output,

the dispenser being arranged for humidifying the wipe.

The invention also encompasses a process to manufacture the dry antibacterial article of the invention, comprising the steps of:
carding dispersible fibers in one direction;
spreading spores onto the carded fibers and
submitting the carded fibers with spores thereon to a. heat treatment to melt the fibers into a nonwoven material.

The invention also relates to a process to manufacture the dry antibacterial article of the invention, comprising the steps of:
carding dispersible fibers in one direction;
submitting the carded fibers to a heat treatment to bond the fibers into a nonwoven material;
spreading spores onto the nonwoven material.

The dry antibacterial article of the invention, its method of use, the dispenser to enable its use and its processes of manufacture are of course linked by a single inventive concept. The effective use of the article of the invention necessitates humidification, which is made possible by the specific dispenser, and a proper release of the spores herein dispersed, which is ensured by its manufacturing methods.

A spore is a stripped-down, dormant form to which bacteria, mainly gram-positive bacteria, can reduce themselves, usually when placed in a situation of lack of nutrients. Other species like fungi can also form spores, but these are out of the scope of the present invention. Spores can remain dormant for extended time periods, even centuries, as they are resistant to harsh conditions like high temperatures, freezing, chemical disinfectants, ultraviolet radiation . . . When the environment becomes more favorable, e.g. in presence of humidity and nutrients, the spores are re-activated to a metabolically active cell. In the case of Bacillus bacteria, the spores are formed from an internal vesicle and are therefore named endospores. The terms endospore and spores are here used indifferently when relating to Bacillus.

The spores are here selected to inhibit the growth of pathogenic bacteria upon reactivation. Pathogenic bacteria are bacteria that can cause an infectious disease, in particular in humans. Several types of pathogenic bacteria are often found in public restrooms, the most abundant being of the genus Escherichia, Staphylococcus and Salmonella, each of these genus having several species. These pathogenic bacteria usually are affected by the presence of lactate or lactic acid, which triggers they decay.

The spores are therefore here selected and prepared from lactate producing bacteria. They can be in particular endospores from the non-pathogenic Bacillus. Lactobacillus would also be interesting to use, but to date, no spore could be prepared/identified from these bacteria. Upon reactivation, the spores of these bacteria will produce lactate or lactic acid, which will inhibit the growth and/or even kill the pathogenic bacteria. The spores should also be selected upon their revival time, as short as possible.

By unprotected and dry spores, it is meant that the spores are not enclosed in microcapsules like the cleansing agent in WO2013171343, and are therefore not surrounded by any humidity. They are directly in contact with the nonwoven material in which they are dispersed.

The dry antibacterial article of the invention can be manufactured. as large sheets, possibly conditioned as rolls, as it is standard practice in the industry of nonwoven materials, or even as precut rolls. The large sheets can be cut to a smaller size or the article of the invention may be directly manufactured at a suitable size to form wipes.

Flushable has here the meaning disclosed in official guidelines from Wastewater Agencies like Edana or Inda, which implies a particular behavior of the article under particular conditions, as will be described below.

By wipes, it is referred to an article, usually square or rectangular, but possibly of any desired shape, having a size in the centimeters range. A wipe generally refers to a single-use, disposable product.

A dispersible nonwoven layer is a thin layer of nonwoven material that has the capacity to degrade in water. In particular, a dispersible nonwoven layer should be suitable to be disposed of in a toilet drain, without blocking the drain or interfering with a sewage pump. The fibers of a dispersible article should become loose within seconds or minutes of being immersed, and progressively solubilize.

The dispersible handling layer is preferably made in a material that is not be permeable to spores and/or humidity for at least the duration of the use of the article, but yet disintegrates when immersed in a large volume of water. A user holding a wipe of the invention by its handling layer is therefore never in contact with any substance present in the nonwoven layer, be it the spores, or any other substance adsorbed by the nonwoven during its use.

Advantageously, when manufactured as a wipe, several dry antibacterial articles can be provided in a dispenser. By dispenser, it is referred to a hard or soft packaging, having an opening or dispensing output, which can be open to remove as article and closed afterwards, in order to ensure the integrity of the articles along the time. A dispenser is arranged to facilitate the removal of one article at a time. Advantageously, when the article is intended to be a transportable personal hygiene article, the dispenser is a small box or package, of, for example, wallet size or smaller, containing a limited number of wipes. The wipes can be suitably folded inside the dispenser so as to be easily removed from the dispenser, and allowing a second wipe to be also easily removed once a first wipe has been taken. For example, the wipes are stored with a so called Z-fold within the dispenser.

The spores spread in the wipes enable a very long shelf life. No problem of loss of activity with time is expected, as it often occurs with wet wipes when they are stored in a packaging not properly sealed.

During the use, just after humidification, spores might be present in the article at different stages of “life”, meaning that dormant spores, spores in reactivation phase, and spores reactivated to bacteria may coexist. The spores released upon use designate one, several or all these forms.

The aqueous liquid may be pure water, or water containing additives, like nutrients or salts suitable to enhance the reactivation rate of the spores. Other possible additives comprise, for example, essential oils or scents.

The humidification of the article may be performed by various methods. For example, the aqueous liquid can be sprayed onto the nonwoven layer. The article may be provided with a bottle of aqueous liquid to be sprayed onto the article. Alternatively, the aqueous liquid could be sprayed directly onto the hard surface to clean, the nonwoven layer absorbing the liquid upon wiping.

A clever solution is to provide article within a dispenser of the invention wherein humidification means are arranged to humidify the article as it is extracted from the dispenser. This is particularly interesting when the dispenser is intended to be easily transported.

An acceptable level of humidity to be deposited onto the hard surface is a level sufficiently high to enable migration/release and reactivation of the spores, but sufficiently low for the comfort of the user of, e.g. a toilet seat. For example, an amount of water containing 0.9% of salts of between 0.05 mL and 0.5 mL deposited on a standard toilet seat before wiping with a nonwoven has been found to leave a level of humidity sufficiently low not to cause discomfort to the toilet user when sitting, while ensuring a correct reactivation of spores.

The invention will be better understood with the following description of several examples, referring to the accompanying drawing on which:

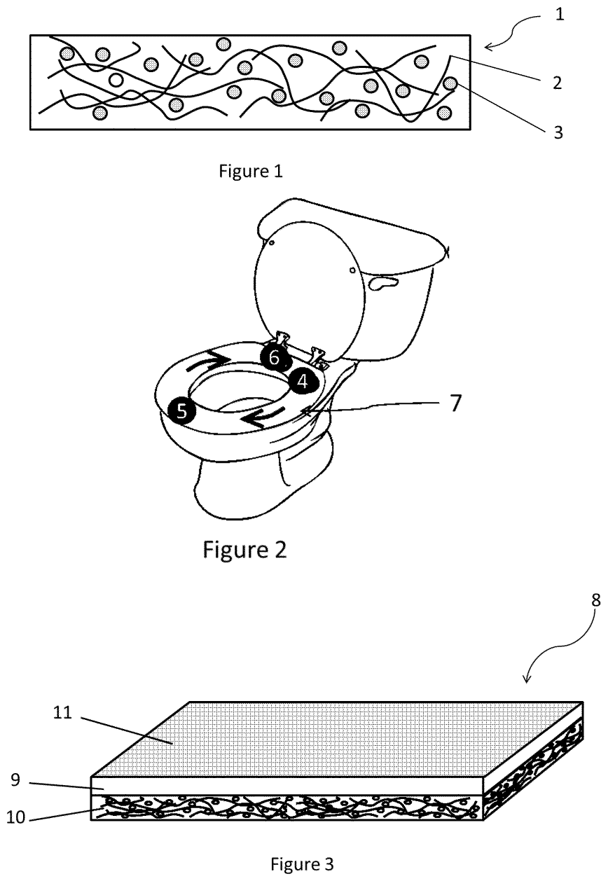

FIG. 1 illustrates a section of a dry antibacterial article of the invention;

FIG. 2 illustrates a wiping pattern according to the method of use of the invention;

FIG. 3 is a three dimensional representation of a wipe made with a dry antibacterial article of the invention;

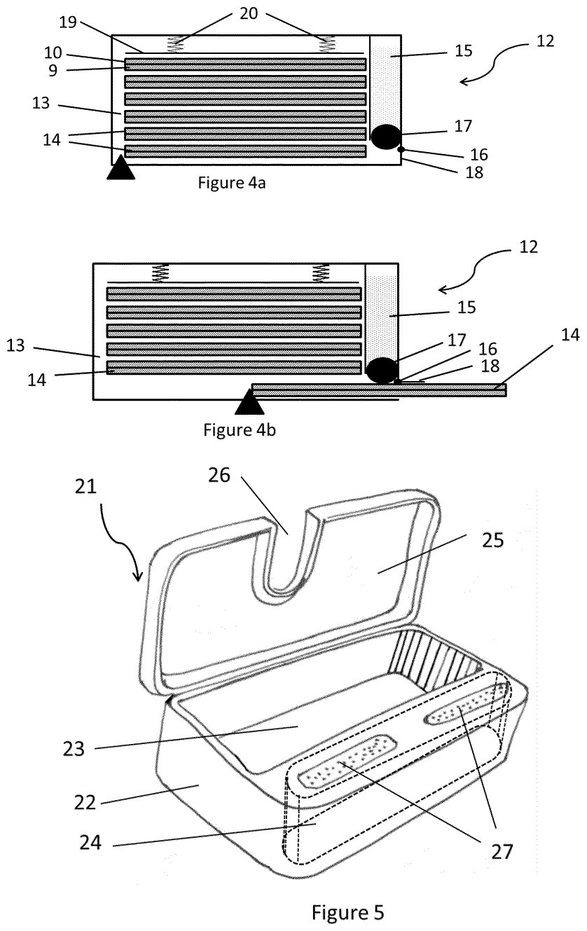

FIG. 4a is a cross sectional view of a full dispenser of the invention;

FIG. 4b illustrates the extraction of a wipe out of the dispenser of FIG. 4a.

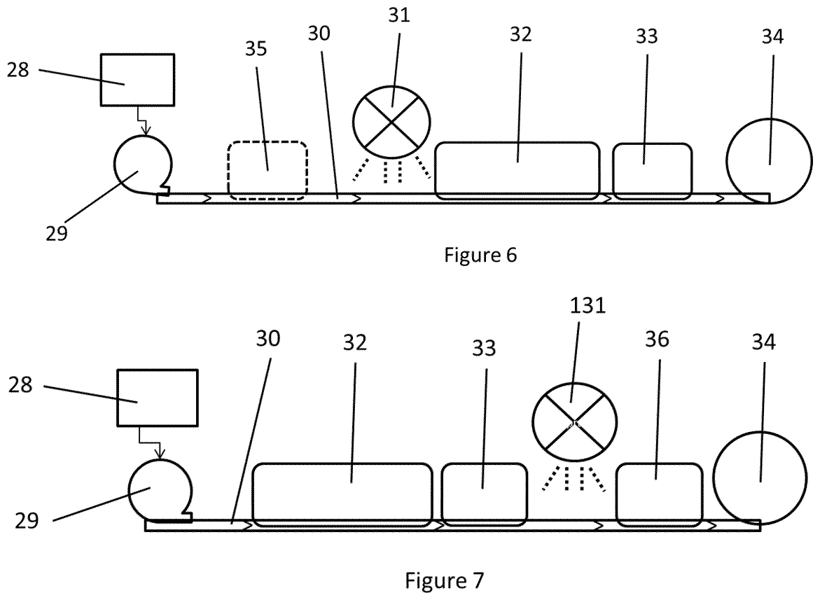

FIG. 5 illustrates another dispenser of the invention;

FIG. 6 is a bloc diagram illustrating a first implementation of the process of the invention, and

FIG. 7 is a bloc diagram illustrating a second implementation of the process of the invention.

In order to be easily transportable and easily usable, the wipes according to the invention are advantageously packaged in a dispenser enables both the long term storage of the wipes, in dry condition, and the humidification of a wipe just before use, to activate the spores dispersed in the nonwoven layer.

Referring to FIGS. 4a and 4b, a dispenser 12 is divided into a dry compartment 13 containing 6 wipes 14, comprising a handling layer 9 and a nonwoven layer 10 with spores therein, horizontally piled up, and a wet compartment 15 filled with an aqueous liquid. The dry compartment 13 is connected to the wet compartment 15 by a cylindrical roll 17 which axis is arranged in the same horizontal plane as the wipes 14. The cylindrical roll 17 is located next to an opening or dispensing output 18 between the dry compartment 13 and the outside of the dispenser 12. The opening is here arranged with a hinge 16, which axis is parallel to the axis of the cylindrical roll 17. A mechanical pusher 16 is also arranged on the base of the dispenser, with a portion situated outside the dispenser and a portion situated inside the dry compartment 13, both portions being connected through a rail opening (not shown) along the base side of the dispenser 12. A holding system is here installed inside the dry compartment 13 consisting of a plate 19, in contact with the wipe 14 at the top of the pile, and two springs 20 connecting the plate 19 to the top inner side wall of the dispenser's dry compartment 13.

The positioning attributes “top”, “base”, “horizontal”, etc. . . . are to be understood as relative attributes between the elements of the dispenser 12, as said dispenser may be carried or used along any orientation.

When it is not in use, the dispenser 12 is in a closed configuration, as on FIG. 4a, the pusher 16 is located at the opposite side of the base from the opening 18, which is in a closed position.

When a user needs a wipe, he actions the pusher 16 laterally, towards the dispensing output 18. This results in the section of the pusher 16 located inside the dispenser 12 pushing the bottom wipe 14 towards the opening 18. The wipe 14 pushes the opening 18 open, by inducing a rotation of the opening 18 around the hinge 16. The wipe 14 can therefore slide out of the dispenser 12, as illustrated on FIG. 4b.

While the wipe 14 is sliding out, it is in contact with the cylindrical roll 17 and induced a rolling movement of the roll 17. This rolling movement induces a displacement of aqueous liquid around the roll 17 and a transfer of liquid onto the part of the nonwoven layer 10 of the wipe 14 in contact with the roll 17. As during the sliding out of the wipe, the full area of the wipe comes in contact with the roll, the full area of the wipe receives some aqueous liquid and is therefore humidified.

The pusher 16 may push the wipe 14 until it is completely outside the dispenser, or may push it only partially. In that case, the user can pull the wipe 14 out of the dispenser 12. Either way, the wipe 14 slides again the rolling cylinder 17 and becomes humidified.

When the wipe 14 is completely out, the opening 18 closes, by back rotation on its hinge 16. The user may push the pusher 16 back to its initial position. Alternatively, a mechanism can be arranged, for example with a spring, to make the pusher 16 come back to its position automatically.

The holding system pushes the pile of wipes 14 towards the base, by releasing tension from the springs 20 on the plate 19, to compensate for removed wipes.

The pusher 16 is only one illustration of a means to force out or extract a wipe out of the dispenser. Several other solutions can. be used to move the wipe out of the dispenser.

Other configurations of dispensers can be envisaged, for example, a larger rechargeable dispenser 21 for domestic use, as illustrated on FIG. 5. A container base 22 is divided in a dry compartment 23 large enough to receive a number of dry antibacterial wipes, and a wet compartment 24 for receiving aqueous liquid, with here two humidifying sections 27, here a spongy material at the interface of the wet compartment 24 and the exterior of the base compartment 22. A lid 25 is arranged with a pulling opening 26.

In the open position, wipes of an article of the invention can be inserted in the dry compartment 23, preferably as pack of wipes with a Z-fold arrangement, or alternatively in the form of a precut roll which would enable to detach single wipes upon pulling. Ideally, the wet compartment comprises an opening, to enable its filling with aqueous liquid. The first wipe can be slightly pulled in such a way that, when the lid 25 is closed, a part of the wipe is visible outside the dispenser.

When the lid 25 is closed, the humidifying sections 27 are covered and thus prevented from drying.

In the closed position, when a user needs a wipe, he pulls an apparent part of the wipe, which comes out of the dispenser 21 by sliding over, at least partly, the humidifying sections 27. The user then wipes the hard surface to be cleaned, and throws the wipe away.

The means to release some aqueous liquid onto the wipe, whether they are the ones described in the examples above, or any other means obvious to a skilled in the art, are preferably arranged to release the optimal amount of liquid, in order to optimize the reactivation of the spores, while depositing an acceptable amount on the hard surface upon wiping. These means typically ensures that the wet compartment remains sealed when no wipe is going out of the dispenser, while enabling some aqueous liquid to be released onto the wipe when the wipe comes out of the dispenser. One may think of a rotative brush, which rotation would be triggered upon exit of the wipe, or a roll dispenser as decribes above, or of the type frequently used for deodorants or liquid glues.

The dispensers described above are convenient for the use of the article of the invention. The user takes a dry antibacterial article out of the dispenser, holding it in a hand. The article has just been humidified while coming out of the dispenser. He then wipes the hard surface to be cleaned with the humidified layer of article, causing the release of spores onto the hard surface, and then throws the article away.

Now that the dispenser has been disclosed, the wipes and their manufacture from an article according to the invention, as well their use will be described.

As disclosed on FIG. 1, a dry antibacterial article 1 comprises a dispersible nonwoven layer made of fibers 2 in which are dispersed unprotected and dry spores 3.

The article of the invention applies the principle of beneficial bacteria targeting pathogenic bacteria that is widely used in our own bodies, on our skin and in our intestines. The challenge to apply this principle to a sheet material, like a nonwoven wipe, is to make sure that the beneficial bacteria will be active at the time of use. There are therefore technical constraints on shelf life, storage conditions, manufacturing process, etc.

Indeed, a wet wipe with bacteria cannot be stored for unlimited time without having over-proliferation of the bacteria impacting the humidity level on the wipe and possibly leading to the death of the beneficial bacteria themselves.

The applicant has cleverly thought of using bacteria under a different form, in particular in their dry form of spore, to overcome the storage problems of wet wipes. A new problem results from the use of the spores, which is reactivating the spores at the right moment, e.g. just before use. This problem was overcome by overseeing humidification solutions to reactivate the spores.

Selection and Preparation of the Spores

Will now be described a process to select and prepare suitable spores for the article of the invention.

The inventors have undergone extensive studies to select suitable bacteria which can:
form spores,
be reactivated to bacteria in a short period of time, and
demonstrate inhibitory activity on pathogenic bacteria.

An unidentified sample possibly containing several types of bacteria was grown in a strict aerobe environment. When typical growths of Bacillus species appeared, pure cultures of each were prepared via standard methods. The pure cultures were analyzed 16S rDNA Sanger sequencing (universal primers 27F-1492R), which is a standard method well known to the skilled in the art to identify bacterial strains.

For the production of spores, an overnight culture of each pure bacteria was first grown in LB (24 h, aerobic, 37° C.). When a stationary culture was obtained, a mixture of salts was added to obtain a final concentration of 0.1% KCl, 0.012% MgSO₄, 1 mM Ca(NO₃)₂, 0.01 mM MnCl₂, 1 μM FeSO₄. The cultures were incubated under the same conditions overnight. Spores, if any, were harvested by centrifugation of each culture.

The spores were sequenced. Four different species were identified:
Bacillus amyloliquefaciens;
Bacillus licheniformis;
Bacillus subtilis, and
Bacillus pumilus.

For each strain, spores were produced in sufficient amount for further tests, using methods well known in the art.

A Bacillus mixture comprising these four species as active bacteria is also prepared for further tests.

The bacteria Lactobacillus rhaomnosus GG was also selected for further tests, despite the fact is does not form spores. It was used as a reference. It could also, for example, be applied on an article of the invention, in combination with the spores.

The antipathogenic activity of the selected strains of bacteria is then assessed. For this purpose, four of the most frequent pathogenic bacteria, found in public restrooms and susceptible of causing gastro-intestinal diseases were selected:
Escherichia coli (LMG2093);
Salmonella enterica subsp. enterica serov. Typhimurium ATCC14028;
Staphylococcus epidermidis (ATCC12228), and
Staphylococcus aureus (ATCC29213).

Several tests were performed to mimic different environments. A well diffusion assay mimics conditions where a beneficial bacteria can release antipathogenic substances in a continuous manner, whereas a streakline assay and a spot assay mimic conditions where a beneficial bacteria release antipathogenic substances when in direct contact with a pathogenic bacteria.

Well Diffusion Assay

A molten agar was inoculated with 500 μl of a pathogenic strain. After the agar solidified, four holes were punched, and filled with 100 μl cell-free supernatant of either the Bacillus mixture or Lactobacillus rhamnosus GG. The assay was repeated for each pathogenic strain. After incubation (24 h, 37° C.), the inhibition zone of growth of the pathogen was measured.

The observed inhibition zone, in millimeters, averaged from triplicate experiments, is given in table 1 below.

TABLE 1

| | | |
|---|---|---|
| | Lactobacillus | |
| Pathogen | rhamnosus GG | Bacillus mixture |

| | | | |
|---|---|---|---|
| Staphylococcus | 0 | mm | 0 mm |
| aureus | | | |
| Staphylococcus | 2.5 | mm | 0 mm |
| epidermidis | | | |
| Salmonella enterica | 2.2 | mm | 0 mm |
| Escherichia coli | 4.78 | mm | 0 mm |

Streakline Assay

On a solid agar plate, a colony of the Bacillus mixture or Lactobacillus rhamnosus GG was inoculated in a straight line from top to bottom. The colony is allowed to grow on incubation (24 h, 37° C.). After overnight incubation, a pathogenic strain was inoculated in a perpendicular line. The pathogenic strain was allowed to grow on incubation (24 h, 37° C.). After incubation the inhibition on the growth of the pathogenic strain was measured. The assay was repeated for each pathogenic strain.

The observed inhibition zone, in millimeters, averaged from triplicate experiments, is given in table 2 below.

TABLE 2

Lactobacillus

| | | |
|---|---|---|
| Pathogen | rhamnosus GG | Bacillus mixture |

| | | | | |
|---|---|---|---|---|
| Staphylococcus | 7.5 | mm | 10 | mm |
| aureus | | | | |
| Staphylococcus | 12 | mm | 11.8 | mm |
| epidermidis | | | | |
| Salmonella enterica | 10 | mm | 0 | mm |
| Escherichia coli | 14.8 | mm | 4.3 | mm |

Spot Assay

First, a single colony of either the Bacillus mixture or Lactobacillus rhamnosus GG was grown on solid LB agar after incubation (overnight, 37° C.). Afterwards, a molten agar was inoculated with 500 μl of a pathogenic strain and poured on top of the colony. After incubation (24 h, 37° C.), the inhibition zone of growth of the pathogen was measured. The assay was repeated for each pathogenic strain.

The observed inhibition zone, in millimetres, averaged from triplicate experiments, is given in table 3 below.

TABLE 3

| | | |
|---|---|---|
| | Lactobacillus | |
| Pathogen | rhamnosus GG | Bacillus mixture |

| | | | | |
|---|---|---|---|---|
| Staphylococcus | 7.8 | mm | 8 | mm |
| aureus | | | | |
| Staphylococcus | 12.2 | mm | 12 | mm |
| epidermidis | | | | |
| Salmonella enterica | 19.5 | mm | 0 | mm |
| Escherichia coli | >20 | mm | 2.2 | mm |

The experiments demonstrate that both the Lactobacillus rhamnosus and the Bacillus mixture effectively inhibit the growth of three of the selected pathogenic bacteria. However, the Bacillus mixture under consideration does not inhibit the Salmonella enterica. The selection of Bacillus species in the mixture could most likely be optimized to also demonstrate inhibition of the Salmonella enterica.

For the intended use of the article of the invention,
the user takes a dry antibacterial article in a hand;
the user wipes the hard surface with the article, causing the release of spores onto the hard surface, and
the user throws the article away,

wherein the dry antibacterial article is humidified just before wiping the hard surface to initiate the reactivation of the spores.

This method ensures the best antibacterial effect of the article on a hard surface.

The use of an article of the invention, made of only one nonwoven layer, to clean a toilet seat is illustrated below with reference to FIG. 2. The use of another article of the invention additionally comprising a paper handling layer is illustrated below with reference to FIG. 3.

Fixation and Release of the Bacillus and Lactobacillus on a Nonwoven

A suspension of bacteria (Bacillus: 7.22×107 cfu; Lactobacillus: 1.69×1011), PVA (3%) and water (2.5 ml) was prepared. This mixture was sprayed on top of the nonwoven material (313 cm²), and subsequently dried in an oven (5 seconds, 180° C.)

After the heat treatment, the samples were tested for the presence of bacteria. A piece measuring 25 cm² was cut out and soaked in 10 ml PBS. The bacterial titer of this PBS was then determined. Less than 1% Lactobacillus could be retrieved, 18.6% of the Bacillus species was still viable on the nonwoven.

Two nonwoven materials were used, a PET nonwoven and a PVA-PLA nonwoven.

The release onto a toilet seat was subsequently assessed in various humidity conditions.

In reference to FIG. 2, a toilet seat 7 was preliminarily sterilized with 70% ethanol. It was then wiped, along a circular movement, with a 25 cm² wipe as previously prepared. The wiping process was performed in three conditions:
i. no transfer liquid was used;
ii. 0.1 mL water was added as a droplet on the toilet seat, where the wiping movement starts, to serve as humidification means of the wipe; and
iii. three droplets of mineral oil were placed around the sterilized toilet seat before wiping.

Sampling the toilet seat on three spots was performed by placing a PBS-soaked paper filter (VWR 516-0812, 55 mm) on each spot. These spots are located at the beginning 4, the middle 5 and the end 6 of the wiping movement on the toilet seat 7. The PBS-soaked filter was removed immediately after placement and placed on solid LB growth medium and incubated (24 h, 37° C.)

For condition i. limited presence of bacteria was identified at the beginning and middle of the wiping movement, indicating some transfer has occurred.

For condition ii., extensive growth of the Bacillus was observed after incubation of the three samples, indicating a very good transfer of the bacteria onto the toilet seat, along the whole wiping movement.

For condition iii., substantial presence of bacteria was identified at the beginning of the wiping movement, but only limited presence of bacteria was observed along the rest of the wiping movement. Moreover, after usage, the toilet seat was covered by an oily residue which was perceived as unpleasant by users.

It was therefore demonstrated that humidification of the wipe, just before use, ensures optimal transfer of the bacteria onto the toilet seat.

Efficiency of a bi-layered article made of a dispersible nonwoven wiping layer and a dispersible paper handling layer.

With reference to FIG. 3, a dry antibacterial wipe 8 comprises a dispersible nonwoven layer 10, wherein spores of a selection of Bacillus spores are dispersed, as prepared above, and a dispersible handling paper layer 9.

To produce a limited number of such wipes 8, a large sheet of nonwoven material, sprayed with a suspension of spores and dried, was cut into pieces of 25 cm². Heat sealable and water soluble paper from Daymark Technologies was cut into pieces of the same size, placed over the nonwoven pieces and sealed theronto by applying heat, by means of a domestic iron. A metal grid is placed between the paper layer and the iron while heating, in order to emboss a pattern to the handling surface 11 of the wipe 8.

Three toilet seats were first sterilized and contaminated with the pathogenic bacteria described above. After 2 h, two toilet seats were wiped, each with a wipe 8, just after humidification of the wipe with 0.5 mL PBS, and following the same wiping pattern as before (FIG. 2). The third toilet seat was left uncleaned for reference.

Samples were taken from each toilet seat on three spots: PBS-soaked paper filters (VWR 516-0812, 55 mm) were placed on spots located at the beginning 4, the middle 5 and the end 6 of the wiping movement on the toilet seat 7 (cf FIG. 2). The PBS-soaked filter was removed immediately after placement and placed in sealed Falcon tubes containing 10 mL PBS. The tubes were shaken 20 minutes to bring all the cells in suspension. Two samples were taken from each falcon tube, which were heated to 80° C. for 15 min to kill all vegetative cells and leave only spores. A dilution array of these two samples were plated and incubated. The Bacillus colonies showing a different morphology from the pathogen colonies, the amount of pathogen present on each spot of each toilet seat could be evaluated.

It was demonstrated that the presence of pathogens on the toilet seats was strongly reduced after the use of the wipe 8, while the presence of Bacillus was significantly increased, indicating both a good transfer of the spores to the toilet seat and an instant effect of these spores. On the uncleaned toilet seat, no Bacillus colonies were identified and the presence of pathogen was reduced to a lesser extent than on the cleaned toilet seats, most likely due to natural death of the pathogens in absence of humidity.

It was also demonstrated that several days after the use of the wipe, the presence of Bacillus could still be observed on the toilet seat, even though the surface has become dry. These indeed have the faculty to turn alternatively into vegetative cells when conditions are favorable and into spores when conditions are less favorable.

Flushability of the Wipe

The wipes made of an article of the invention are single use wipes that are suitable to be thrown away, in particular flushed in a toilet. The wipes should therefore readily disperse in order not to damage the drainline or any component of the toilet evacuation system. The validation of the flushable character of wipes made of the article of the invention is detailed below.

The differentiation of flushable and non-flushable nonwoven products obeys to strict guidelines established by Wastewater agencies, like Edana in Europe and INDA in the US. The technical flushability assessment comprises 7 tests which are
the Toilet and Drainline Clearance Test (FG501), to determine the likelihood that a product will successfully clear toilet and building drainage lines;
the Slosh Box Disintegration Test (FG502), to assess the potential for a product to disintegrate when subjected to mechanical agitation in water or wastewater;
the Household Pump Test (FG503), to assess the compatibility of a product with household sewage ejector pump systems to ensure that the product does not clog, accumulate within or otherwise interfere with normal system operation under high usage conditions;
the Settling Test (FG504), to assess whether a product settles in sumps, septic tanks, onsite aerobic systems and settling chambers that are associated with pump stations and municipal wastewater treatment plants;
the Aerobic Biodisintegration/Biodegradation Test (FG505), to assess the potential for a product to biologically degrade under aerobic conditions typically found in sewers as well as onsite and municipal wastewater treatment systems;
the Anaerobic Biodisintegration/Biodegradation Test (FG506),to assess the potential for a product to biologically degrade under anaerobic conditions typically found in sewers as well as onsite and municipal wastewater treatment systems; and
the Municipal Sewage Pump Test (FG507), to assess the compatibility of products with small municipal sewage pump systems.

The description and method of each of these tests is available on www.edana.org/industry-initiatives/flushability.

25 cm² wipes, as prepared previoulsy, were used to conduct each test. The results are summarized in table 4, showing that the wipe is allowable to claim flushability.

TABLE 4

| Test | Requirement | Result |
|---|---|---|
| FG501 | The travel distance of the | The center of gravity of |
| | Centre of Mass of the | the wipe does not descend, |
| | flushed material in the | the 11^{th} flush |
| | drain line does not | makes the 20 m drain |
| | consistently decrease over | free - pass |
| | the course of 5 | |
| | consecutive flushes | |
| FG502 | >25% of an article's | >54% of all 9 wipes pass |
| | initial dry mass must pass | through the sieve - pass |
| | through the sieve, and | |
| | this condition must be met | |
| | for 80% or more of the | |
| | replicate articles tested. | |
| FG503 | The product must not cause | At the end of the test (7 |
| | the system to stop | days), 3.2 nonwoven |
| | functioning at any point | residues were counted in |
| | during the test, | the bin (12 wipes/day |
| | AND | flushed) - pass |
| | the average number of | |
| | articles remaining in the | |
| | basin at the end of days 2 | |
| | through 6 must not exceed | |
| | the number of articles | |
| | loaded on a daily basis. | |
| FG504 | The average settling | All 10 wipes satisfy the |
| | velocity for the articles | requirements - pass |
| | that settle must exceed | |
| | 0.1 cm/sec and at least 95% | |
| | of the total articles | |
| | tested must settle | |
| | AND | |
| | at least 95% of the | |
| | articles tested must not | |
| | become sufficiently | |
| | buoyant to rise more than | |
| | 30 cm from the bottom | |
| | within 24 hrs. | |
| FG507 | The average percent power | The average power increase |
| | increase of the pump over | is 6.6% - pass |
| | baseline for the 5 runs | |
| | must not exceed 15%. | |

When the article is used to clean a toilet seat, the article can advantageously be flushed, i.e. thrown away in the water of the toilet.

A small scale manufacturing method of wipes made with dry antibacterial articles according to the invention has been described, with a simple manufacturing process, starting from already manufactured nonwoven material.

The adhesion of the spores to the nonwoven layer in the dry article, along to their releasing potential when humidified, are critical features to obtain the desired antibacterial activity. These features have been optimized by the applicant by setting up an innovative manufacture process.

In reference to FIG. 6, in a first step, raw fibers contained in a bale opener 28 are introduced in a carding machine where they are carded in one direction into a web. The web in then moved along the line by means of a conveyor belt 30. In a second step, spores are dispersed on the web of carded fibers by means of spraying equipment 31. In a further step, the fibers of the web are bonded in an oven 32 before going through a cooling zone 33. The nonwoven material is finally rolled up by a rolling up equipment 34. An optional of needling using module 35 is here inserted between the carding and the spraying steps.

Apart from the step of spraying spores with equipment 31, the manufacturing process disclosed on FIG. 6 comprises steps using equipment well known to a person skilled in the art. Inserting equipment on an existing manufacturing line is however not necessarily easy. Constraints of space apply, as well as constraints of speed to synchronize the various steps.

According to the manufacturing process of the invention, spores are applied on carded fibers, before the bonding step leading to the manufacture of the nonwoven material. This process cleverly makes profit of the resistance of the spores to a heat treatment, to disperse them during the manufacture of the nonwoven material itself. This means a significant gain of time in the process, as well as a better dispersion of the spores in the nonwoven compared with a conventional process where the spores would be applied to the nonwoven material after its manufacture.

Depending on the thickness expected for the nonwoven material, several layers of carded fibers, of a same or of different compositions, can be overlaid before the dispersion of the spores, using techniques and equipment well known to a person skilled in the art. Using several carding machines in parallel, usually up to three, allows to work at high speed. The resulting webs are then overlapped before bonding, or before needling in case it is implemented in the process. This also presents the advantage of being able to combine the different properties of several fiber blends.

Needling results in entangling or mixing up the fibers and is especially recommended when more than one carding machine is used. It enables to obtain a better adhesion of the web layers, by entangling the fibers. Hydroentanglement could also be used instead of or additionally to needling.

The spores can be dispersed onto the carded fibers by wet spraying, the process then comprises a step of dissolving the spores just before spraying. Spraying then occurs just before, i.e. a few milliseconds to a minute before, the heat treatment, in order not to let the spores enough time to reactivate. The heat treatment then has a double effect of drying the spores and melting the fibers to a nonwoven material.

The spores could also be dispersed onto the carded fibers in a dry form, by powder scattering, using for example a powder spray commercialized by the company WEKO. The timing of dispersion of the spores would, in that case, be less crucial as no humidity would be present, eliminating the risk of reactivation of the spores.

Applying the spores by spraying or by powder scattering gives, in the end, the same nonwoven article wherein spores are dispersed.

Any technology typically used for coating nonwoven can be used for applying the spores, and in particular, systems from WEKO, like the “WEKO-Fluid-Application-System (WFA)”.

Bonding of the fibers to finalize the nonwoven layer can be performed using different techniques, like mechanical or chemical bonding. In the present case, bonding preferably includes a step of thermal bonding, either alone, or in combination with another technique. Preferably, the nonwoven of the invention is a drylaid thermobonded nonwoven (through air bonded).

Thermo-bonding ovens are available on the market, like flat belt ovens, tumble oven or omega ovens, manufactured, for example, by WEKO. Such oven could even integrate, at their entry, a spraying unit.

The heat treatment usually applied to the carded fibers ranges between temperatures of 30° C. and 250° C., preferably between 130° C. and 140° C., depending on the nature of the fibers and the temperature needed for bonding, the line speed and the temperature needed to evaporate the spore dissolving solution in case of wet spraying.

The residence time of the material layer in the oven, depending on the speed of the manufacturing line, is comprised between a second and a few minutes, which allows any humidity introduced during a spraying step to evaporate.

The manufacturing process resulting in the production of a continuous layer of nonwoven material, the nonwoven layer, whether it has been adhered to a handling layer or not, can be rolled as it reaches the end of the manufacturing line. Optionally, the nonwoven layer can be precut into smaller entities before being rolled up. Alternatively, the continuous nonwoven layer can be cut into smaller pieces and conditioned as piled up packs.

In some cases, it may not be possible to insert spraying equipment before the bonding ovens. In other cases, it could be interesting to spray onto the nonwoven, additionally to spores, some living bacteria which would not bear the thermal treatment during bonding. It is then possible to proceed with the spraying step after the bonding of the nonwoven, as is illustrated in FIG. 7.

In that case, the spray equipment 131 is installed after the cooling zone 33. In case of wet spraying, it is necessary to add a step of drying in a further drying zone 36. The characteristics of the other pieces of equipment remain the same as described above. Though this order of steps may result in a slightly less good dispersion of the spores than when the spores are dispersed before bonding, it results however in a product having the wiping and disinfecting qualities described above.

In some cases where spraying the spores cannot be done at the same manufacturing site as the manufacture of the nonwoven, it is even possible to unroll a roll of nonwoven, disperse the spores onto the nonwoven and roll up the nonwoven containing dispersed spores, or recondition it in any suitable manner.

The processes of the invention can comprise a further step of adhering the nonwoven layer to at least another layer of material, in particular to a handling layer. Adhesion can be obtained by mechanical means of by using adhering intermediate substances. Assembling the nonwoven layer containing spores with a handling layer can be performed on the same manufacturing line or on a separate line, or even at a different facility.

The nonwoven layer, whether it has been adhered to a handling layer or not, which has been conditioned as a roll may further be processed, cut and/or reconditioned to any suitable form for the purpose of the invention.

The fibers used for the purpose of the invention may consist of one type, or may be a blend of several types of fibers. At least one of these fibers should have a melting temperature within the temperature range of the heat treatment, to ensure proper bonding and resistance of the nonwoven layer.

This process is applicable to many types of fibers. For example, for manufacturing a dispersible nonwoven layer, blends comprising synthetic fibers like, but not limited to, polyolefins, polyesters, polylactates, polyvinylacohols and viscose, and/or natural fibers or biocomponent fibers like, but not limited to, cellulose, can be used.

The fiber decitex can vary between 0.3 dt and 64 dt, preferably between 2.2 dt and 6.7 dt.

A dispersible handling layer can be made from, for example, but not limited to, water soluble paper, water soluble plastic PVA, water soluble polyester, a water soluble coating like a graphene film resin or a reusable injection molded part. This handling layer is preferably impermeable.

Interestingly, monolayer or multilayer paper can be used as handling layer. Paper can confer to the article a certain rigidity, depending on the type and thickness of the paper used. Paper is also advantageously printable, for aesthetic purpose. Biodegradable paper is readily available at low cost. However, any other dispersible material, having similar properties, may also be used as handling layer.

1. A dry antibacterial article comprising:
a dispersible nonwoven layer, wherein unprotected and dry bacterial spores are spread within the nonwoven layer, said spores being selected to specifically inhibit the growth of pathogenic bacteria upon reactivation.

2. The article according to claim 1, wherein the article is flushable.

3. The article according to claim 1, further comprising a dispersible handling layer.

4. The article according to claim 1, wherein the article has been formed into a roll.

5. The article according to claim 4, wherein the roll has been precut into wipes.

6. The article according to claim 1, wherein the article is a wipe.

7. (canceled)

8. The article according to claim 1, wherein the spores are spores of Bacillus bacteria.

9. The article according to claim 1, wherein the nonwoven layer comprises at least one fiber of the group consisting of polyolefins, polyesters, polylactates, polyvinylacohols, viscose and cellulose.

10. (canceled)

11. (canceled)

12. A method of using the dry antibacterial article according to claim 1, by which a user cleans a hard surface, the method comprising:
the user takes the dry antibacterial article in a hand;
the dry antibacterial article is humidified to initiate the reactivation of the spores,
the user wipes the hard surface with the article causing the release of spores onto the hard surface, and
the user throws the article away.

13. (canceled)

14. A dispenser of wipes according to claim 5, the dispenser comprising:
a dry compartment comprising at least one wipe,
a wet compartment, comprising an aqueous liquid,
means arranged to release an aqueous liquid onto the wipe, and a dispensing output,
the dispenser being arranged for humidifying the wipe at the output of the dispenser.

15. The dispenser according to claim 14, wherein the means to release aqueous liquid are arranged between the dry compartment and the dispensing output.

16. (canceled)

17. A method of manufacturing the dry antibacterial article according to claim 1, the method comprising:
carding dispersible fibers in one direction;
spreading spores onto the carded fibers; and
submitting the carded fibers with spores thereon to a heat treatment to bond the fibers into a nonwoven material.

18. The method according to claim 17, wherein the spores are spread by spraying onto the carded dispersible fibers a solution of spores and wherein the heat treatment further evaporates the liquid of the solution sprayed.

19. The method according to claim 18, further comprising dissolving the spores just before spraying.

20. The method according to claim 18, wherein spraying occurs just before the heat treatment.

21. The method according to claim 17, wherein the spores are spread by scattering the spores in powder form.

22. (canceled)

23. A method of manufacturing the dry antibacterial article according to claim 1, the method comprising:
carding dispersible fibers in one direction;
submitting the carded fibers to a heat treatment to bond the fibers into a nonwoven material; and
spreading spores onto the nonwoven material.

24. The method according to claim 13, wherein:
the spores are spread by spraying onto the nonwoven material a solution of spores; and
the method further comprises subsequently drying the sprayed nonwoven material.

25. The method according to claim 24, further comprising dissolving the spores just before spraying.

26. The method according to claim 23, wherein the spores are spread by scattering the spores in powder form.

27. (canceled)
